(19) United States
(12) Patent Application Publication
(10) Pub. No.: US 2009/0299466 A1
(43) Pub. Date: Dec. 3, 2009

(54) Local Delivery of Matrix Metalloproteinase Inhibitors

(75) Inventor: Ayala Hezi-Yamit, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/131,652

(22) Filed: Jun. 2, 2008

(51) Int. Cl. A61F 2/82

(52) U.S. Cl. 623/1.42; 623/1.46

(57) ABSTRACT

Disclosed are medical devices and methods for the local delivery and treatment of vascular conditions. The methods and treatments involve local delivery of at least one matrix metalloproteinase inhibitor. The vascular conditions described herein include plaque rupture, aneurysm, stenosis, restenosis, atherosclerosis and combinations thereof.

### FIELD OF THE INVENTION

The present invention relates to the local delivery of matrix metalloproteinase inhibitors for the treatment of aneurism, restenosis and atherosclerotic plaque stabilization.

### BACKGROUND OF THE INVENTION

Matrix metalloproteinases (MMPs) are a subclass of endopeptidases and serve to degrade certain extracellular proteins. MMPs have roles in cell proliferation, migration, differentiation, angiogenesis, apoptosis, and host defense. MMPs are secreted by inflammatory cells, monocytes and neutrophils, typically in response to inflammation. The secretion of MMPs is the leading cause of extracellular matrix degradation.

Aneurysms are commonly characterized by inflammation and ballooning of a vessel wall, most commonly the aorta. An aneurysm commonly results from a weakening of the vessel wall and is characterized by destruction of extracellular matrix as a result of the inflammatory process. The inflammation of the aneurysm can cause it to grow large enough to rupture, upon which death is immanent.

Atherosclerosis is a condition in which plaque on vessel walls ruptures leading to re-stabilization of the ruptured region of the vessel. Inflammation of the rupture site, the lesion, can lead to proliferation of tissue and eventual stenosis of the vessel. In addition, destruction of the extracellular matrix that cups the atherosclerotic lesion can lead to further plaque rupture and possibly a fatal thrombotic event.

Restenosis is characterized by an inflammatory response to the treatment of a previously stenosis of a vessel. The result of the inflammatory response is commonly tissue proliferation around the angioplasty site. The proliferation of tissue can result in the re-closure of the vessel.

As such, methods are needed to reduce the destruction of extracellular matrix, thereby reducing the prevalence of the conditions above.

### SUMMARY OF THE INVENTION

Disclosed herein are medical devices and methods for the local delivery and treatment of vascular conditions. The methods and treatments involve local delivery of at least one matrix metalloproteinase inhibitor. The vascular conditions described herein include plaque rupture, aneurysm, stenosis, restenosis, atherosclerosis and combinations thereof.

Described herein is a medical device for treating a vascular condition comprising: a stent; at least one polymer; and a therapeutically effective amount of at least one matrix metalloproteinase inhibitor; wherein the stent is adapted to deliver the matrix metalloproteinase inhibitor to a tissue within a mammal suffering from a vascular condition. In one embodiment, the matrix metalloproteinase inhibitor comprises 3-(N-hydroxycarbamoyl)-2(R)-isobutylpropionyl-L-tryptophan methylamide. In another embodiment, the matrix metalloproteinase inhibitor comprises 2-[4-(4-methoxybenzamido)phenylsulfonamido]-6-(4-morpholinyl)-4-hexynoic acid.

In one embodiment, the 3-(N-hydroxycarbamoyl)-2(R)-isobutylpropionyl-L-tryptophan methylamide can be present in an amount of 1 to 1000 μg. In another embodiment, the 2-[4-(4-methoxybenzamido)phenylsulfonamido]-6-(4-morpholino)-4-hexynoic acid can be present in an amount of from about 1 to about 1000 μg.

In one embodiment, the vascular condition being treated is selected from the group consisting of plaque rupture, aneurysm, stenosis, restenosis, atherosclerosis, and combinations thereof.

In another embodiment, the polymer is selected from the group consisting of polyurethanes, silicones, polyolefins, polyisobutylene, ethylene-alphaolefin copolymers, acrylic polymers and copolymers, ethylene-co-vinylacetate, polybutylmethacrylate, vinyl halide polymers and copolymers, polyvinyl chloride; polyvinyl ethers, polyvinyl methyl ether, polyvinylidene halides, polyvinylidene fluoride, polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate; cellophane, cellulose nitrate, cellulose propionate, cellulose ethers, carboxymethyl cellulose, poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes, biomolecules such as fibrin, fibrinogen, cellulose, starch, collagen and hyaluronic acid, and combinations thereof.

In one embodiment, the stent comprises a ratio of matrix metalloproteinase inhibitor to polymer. In one embodiment, the ratio is between about 1:1 and 1:20.

Described herein is a vascular stent comprising a polymeric coating having a therapeutically effective amount of at least one matrix metalloproteinase inhibitor. In one embodiment, the stent further comprises a primer coat. In one embodiment, the matrix metalloproteinase inhibitor comprises 3-(N-hydroxycarbamoyl)-2(R)-isobutylpropionyl-L-tryptophan methylamide. In another embodiment, the matrix metalloproteinase inhibitor comprises 2-[4-(4-methoxybenzamido)phenylsulfonamido]-6-(4-morpholinyl)-4-hexynoic acid.

In one embodiment, the polymeric coating comprises at least one polymer selected from the group consisting of polyurethanes, silicones, polyolefins, polyisobutylene, ethylene-alphaolefin copolymers, acrylic polymers and copolymers, ethylene-co-vinylacetate, polybutylmethacrylate, vinyl halide polymers and copolymers, polyvinyl chloride; polyvinyl ethers, polyvinyl methyl ether, polyvinylidene halides, polyvinylidene fluoride, polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate; cellophane, cellulose nitrate, cellulose propionate, cellulose ethers, carboxymethyl cellulose, poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes, biomolecules such as fibrin, fibrinogen, cellulose, starch, collagen and hyaluronic acid, and combinations thereof.

In one embodiment, the stent comprises a ratio of matrix metalloproteinase inhibitor to polymer. In one embodiment, the ratio is between about 1:1 and about 1:20.

Also described herein is a method of treating a vascular condition in a mammal comprising local delivery of at least one matrix metalloproteinase inhibitor to a mammal suffering from a vascular condition selected from the group consisting of plaque rupture, aneurysm, stenosis, restenosis, atherosclerosis, and combinations thereof. In one embodiment, the matrix metalloproteinase inhibitor is delivered using a vascular stent.

In one embodiment, the matrix metalloproteinase inhibitor comprises 3-(N-hydroxycarbamoyl)-2(R)-isobutylpropionyl-L-tryptophan methylamide. In another embodiment, the matrix metalloproteinase inhibitor comprises 2-[4-(4-methoxybenzamido)phenylsulfonamido]-6-(4-morpholinyl)-4-hexynoic acid.

Also described herein is a method for inhibiting restenosis comprising providing a vascular stent having a coating comprising an therapeutically effective amount of a bioactive agent selected from the group consisting of 3-(N-hydroxycarbamoyl)-2(R)-isobutylpropionyl-L-tryptophan methylamide, 2-[4-(4-methoxybenzamido)phenylsulfonamido]-6-(4-morpholinyl)-4-hexynoic acid, and combinations thereof.

### DEFINITION OF TERMS

Bioactive Agent: As used herein “bioactive agent” shall include any drug, pharmaceutical compound or molecule having a therapeutic effect in an animal. Exemplary, non-limiting examples include anti-proliferatives including, but not limited to, macrolide antibiotics including FKBP 12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, and transforming nucleic acids. Bioactive agents can also include cytostatic compounds, chemotherapeutic agents, analgesics, statins, nucleic acids, polypeptides, growth factors, and delivery vectors including, but not limited to, recombinant micro-organisms, and liposomes.

Exemplary FKBP 12 binding compounds include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid) and zotarolimus (ABT-578). Additionally, and other rapamycin hydroxyesters may be used in combination with the terpolymers described herein.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Biodegradable: As used herein “biodegradable” refers to a polymeric composition that is biocompatible and subject to being broken down in vivo through the action of normal biochemical pathways. From time-to-time bioresorbable and biodegradable may be used interchangeably, however they are not coextensive. Biodegradable polymers may or may not be reabsorbed into surrounding tissues, however, all bioresorbable polymers are considered biodegradable. Biodegradable polymers are capable of being cleaved into biocompatible byproducts through chemical- or enzyme-catalyzed hydrolysis.

Nonbiodegradable: As used herein “nonbiodegradable” refers to a polymeric composition that is biocompatible and not subject to being broken down in vivo through the action of normal biochemical pathways.

Not Substantially Toxic: As used herein “not substantially toxic” shall mean systemic or localized toxicity wherein the benefit to the recipient is out-weighted by the physiologically harmful effects of the treatment as determined by physicians and pharmacologists having ordinary skill in the art of toxicity.

Pharmaceutically Acceptable: As used herein “pharmaceutically acceptable” refers to all derivatives and salts that are not substantially toxic at effective levels in vivo.

### DETAILED DESCRIPTION OF THE INVENTION

Amongst other functions, matrix metalloproteinases (MMPs) degrade certain extracellular proteins. The secretion and function of MMPs are the leading cause of extracellular matrix degradation. The destruction of an extracellular matrix is a leading cause of vascular conditions such as, but not limited to, aneurysm, stenosis, restinosis and atherosclerosis. Therefore, methods need to be developed to either reduce the destruction of the extracellular matrix or reduce the activity of MMPs, which in turn can reduce extracellular matrix destruction.

Matrix metalloproteinase inhibitors (MMPIs) can be endogenous, such as tissue inhibitors of metalloproteinases (TIMPs) or can by synthetic. Synthetic inhibitors commonly contain a chelating group to bind to the catalytic zinc group of the metalloproteinases. Common chelating groups include, but are not limited to thiols, carboxylates, phosphyols, and hydroxamates. The binding of the synthetic inhibitor reduces the activity of the MMP to which it is bounded.

Described herein are MMP inhibitors useful for local delivery to vascular areas in mammals susceptible to, or effected by aneurysm, atherosclerosis, plaque rupture, stenosis and/or restenosis. Local, site specific delivery of MMP inhibitors can reduce the destruction of the extracellular matrix, by inhibiting enzymes belonging to the MMP family, which rapidly degrade the extracellular matrix. Local delivery can also prolong the beneficial effects of the MMP inhibitor within the treated vessel and minimize systemic exposure to the drug. The main benefits of local delivery of an MMP inhibitor would be comprised of local treatment of aneurysm, atherosclerosis, plaque rupture, stenosis and/or restenosis.

The MMP inhibitor used for local delivery can be any endogenous or synthetic MMP inhibitor. In some embodiments, the inhibitor can be any derivative, salt, prodrug or combination thereof, of the MMP inhibitor.

In one embodiment, the MMP inhibitor can be 3-(N-hydroxycarbamoyl)-2(R)-isobutylpropionyl-L-tryptophan methylamide, commonly named ilomastat. Ilomastat has the structure shown below.

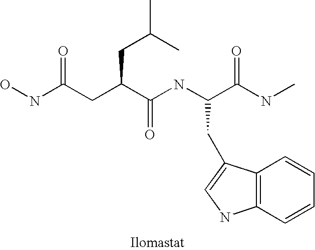

In another embodiment, the MMP inhibitor can be 2-[4-(4-methoxybenzamido)phenylsulfonamido]-6-(4-morpholinyl)-4-hexynoic acid, commonly named PG-530742. PG-530742 has the structure shown below.

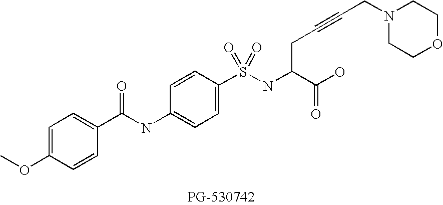

It will be understood by those skilled in the art, that ilomastat and PG-530742 are but two of many pharmaceutically acceptable MMP inhibitors. Many other pharmaceutically acceptable forms can be synthesized and are still considered to be within the scope of the present description. Moreover, many derivatives are also possible that do not affect the efficacy or mechanism of action of the MMP inhibitors. Therefore, the present description is intended to encompass 3-(N-hydroxycarbamoyl)-2(R)-isobutylpropionyl-L-tryptophan methylamide (ilomastat), 2-[4-(4-methoxybenzamido)phenylsulfonamido]-6-(4-morpholinyl)-4-hexynoic acid (PG-530742), and pharmaceutically acceptable derivatives, salts, prodrugs, and combinations thereof.

The MMP inhibitors discussed herein may be added to implantable medical devices. The MMP inhibitors may be incorporated into the polymer coating applied to the surface of a medical device or may be incorporated into the polymer used to form the medical device. The MMP inhibitor may be coated to the surface with or without a polymer using methods including, but not limited to, precipitation, coacervation, and crystallization. The MMP inhibitor may be bound covalently, ionically, or through other intramolecular interactions, including without limitation, hydrogen bonding and van der Waals forces.

The medical devices used may be permanent medical implants, temporary implants, or removable devices. For example, and not intended as a limitation, the medical devices may include stents, catheters, micro-particles, probes, and vascular grafts.

In one embodiment, stents may be used as a drug delivery platform. The stents may be vascular stents, urethral stents, biliary stents, or stents intended for use in other ducts and organ lumens. Vascular stents, for example, may be used in peripheral, neurological, or coronary applications. The stents may be rigid expandable stents or pliable self-expanding stents. Any biocompatible material may be used to fabricate stents, including, without limitation, metals and polymers. The stents may also be bioresorbable. In one embodiment, vascular stents are implanted into coronary arteries immediately following angioplasty. In another embodiment, vascular stents are implanted into the abdominal aorta to treat an abdominal aneurysm.

In one embodiment, metallic vascular stents are coated with one or more MMP inhibitors, the compounds of ilomastat or PG-530742. The MMP inhibitor may be dissolved or suspended in any carrier compound that provides a stable, un-reactive environment for the inhibitor. The stent can be coated with an MMP inhibitor coating according to any technique known to those skilled in the art of medical device manufacturing. Suitable, non-limiting examples include impregnation, spraying, brushing, dipping and rolling. After the MMP inhibitor is applied to the stent, it is dried leaving behind a stable MMP inhibitor delivering medical device. Drying techniques include, but are not limited to, heated forced air, cooled forced air, vacuum drying or static evaporation. Moreover, the medical device, specifically a metallic vascular stent, can be fabricated having grooves or wells in its surface that serve as receptacles or reservoirs for the MMP inhibitors described herein.

The effective amount of MMP inhibitor used can be determined by a titration process. Titration is accomplished by preparing a series of stent sets. Each stent set will be coated, or contain different dosages of MMP inhibitor. The highest concentration used will be partially based on the known toxicology of the compound. The maximum amount of drug delivered by the stents will fall below known toxic levels. The dosage selected for further studies will be the minimum dose required to achieve the desired clinical outcome. In one embodiment, the desired clinical outcome is defined as a site specific decrease in MMP activity or decrease in extracellular matrix destruction.

In another embodiment, the MMP inhibitor is precipitated or crystallized on or within the stent. In yet another embodiment, the MMP inhibitor is mixed with a suitable biocompatible polymer (bioerodable, bioresorbable, or non-erodable). The polymer-MMP inhibitor blend can then be used to produce a medical device such as, but not limited to, stents, grafts, micro-particles, sutures and probes. Furthermore, the polymer-MMP inhibitor blend can be used to form controlled-release coatings for medical device surfaces. For example, and not intended as a limitation, the medical device can be immersed in the polymer-MMP inhibitor blend, the polymer-MMP inhibitor blend can be sprayed, or the polymer-MMP inhibitor blend can be brushed onto the medical device. In another embodiment, the polymer-MMP inhibitor blend can be used to fabricate fibers or strands that are embedded into the medical device or used to wrap the medical device.

In one embodiment, the polymer chosen must be a polymer that is biocompatible and minimizes irritation to the vessel wall when the medical device is implanted. The polymer may be either a biostable or a bioabsorbable polymer depending on the desired rate of release or the desired degree of polymer stability. Bioabsorbable polymers that can be used include poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid.

Also, biostable polymers with a relatively low chronic tissue response such as polyurethanes, silicones, and polyesters could be used and other polymers could also be used if they can be dissolved and cured or polymerized on the medical device such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The polymer coatings or medical devices formed from polymeric material discussed herein may be designed with a specific dose of MMP inhibitor. That dose may be a specific weight of inhibitor added or a MMP inhibitor to polymer ratio. In one embodiment, the medical device can be loaded with from about 1 to about 1000 μg of MMP inhibitor; in another embodiment, from about 5 μg to about 500 μg; in another embodiment from about 10 μg to about 250 μg; in another embodiment, from about 15 μg to about 150 μg. A ratio may also be established to describe how much MMP inhibitor is added to the polymer that is coated to or formed into the medical device. In one embodiment a ratio of about 1 part MMP inhibitor to about 1 part polymer may be used; in another embodiment, between about 1:1 and about 1:5; in another embodiment, between about 1:1 and about 1:9; in another embodiment, between about 1:1 and about 1:20.

In addition to the site specific delivery of MMP inhibitors, the implantable medical devices discussed herein can accommodate one or more additional bioactive agents. The choice of bioactive agent to incorporate, or how much to incorporate, will have a great deal to do with the polymer selected to coat or form the implantable medical device. A person skilled in the art will appreciate that hydrophobic agents prefer hydrophobic polymers and hydrophilic agents prefer hydrophilic polymers. Therefore, coatings and medical devices can be designed for agent or agent combinations with immediate release, sustained release or a combination of the two.

Exemplary, non limiting examples of bioactive agents include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers described herein.

### EXAMPLES

### Providing a Metallic Surface with an MMP Inhibitor-Eluting Coating

The following Examples are intended to illustrate a non-limiting process for coating metallic stents with an MMP inhibitor. One non-limiting example of a suitable metallic stent is the Medtronic/AVE S670™ 316L stainless steel coronary stent.

### Example 1

### Metal Stent Cleaning Procedure

Stainless steel stents were placed a glass beaker and covered with reagent grade or better hexane. The beaker containing the hexane immersed stents was then placed into an ultrasonic water bath and treated for 15 minutes at a frequency of between approximately 25 to 50 KHz. Next the stents were removed from the hexane and the hexane was discarded. The stents were then immersed in reagent grade or better 2-propanol and vessel containing the stents and the 2-propanol was treated in an ultrasonic water bath as before. Following cleaning the stents with organic solvents, they were thoroughly washed with distilled water and thereafter immersed in 1.0 N sodium hydroxide solution and treated at in an ultrasonic water bath as before. Finally, the stents were removed from the sodium hydroxide, thoroughly rinsed in distilled water and then dried in a vacuum oven over night at 40° C. After cooling the dried stents to room temperature in a desiccated environment they were weighed their weights were recorded.

### Example 2

### Coating a Cleans Dried Stent Using a Drug/polymer System

In the following Example, ethanol is chosen as the solvent of choice. The MMP inhibitor is 3-(N-hydroxycarbamoyl)-2(R)-isobutylpropionyl-L-tryptophan methylamide (ilomastat), herein referred to as ilomastat. Both the polymer and ilomostat are freely soluble in ethanol. Persons having ordinary skill in the art of polymer chemistry can easily pair the appropriate solvent system to the polymer-drug combination and achieve optimum results with no more than routine experimentation.

250 mg of ilomostat is carefully weighed and added to a small neck glass bottle containing 2.8 ml of ethanol. The ilomastat-ethanol suspension is then thoroughly mixed until a clear solution is achieved.

Next 250 mg of polycaprolactone (PCL) is added to the ilomastat-ethanol solution and mixed until the PCL dissolved forming a drug/polymer solution.

The cleaned, dried stents are coated using either spraying techniques or dipped into the drug/polymer solution. The stents are coated as necessary to achieve a final coating weight of between approximately 10 μg to 1 mg. Finally, the coated stents are dried in a vacuum oven at 50° C. over night. The dried, coated stents are weighed and the weights recorded.

The concentration of drug loaded onto (into) the stents is determined based on the final coating weight. Final coating weight is calculated by subtracting the stent's pre-coating weight from the weight of the dried, coated stent.

### Example 3

### Coating a Clean, Dried Stent Using a Sandwich-Type Coating

A cleaned, dry stent is first coated with polyvinyl pyrrolidone (PVP) or another suitable polymer followed by a coating of ilomastat. Finally, a second coating of PVP is provided to seal the stent thus creating a PVP-ilomastat-PVP sandwich coated stent.

### The Sandwich Coating Procedure:

100 mg of PVP is added to a 50 mL Erlenmeyer containing 12.5 ml of ethanol. The flask was carefully mixed until all of the PVP is dissolved. In a separate clean, dry Erlenmeyer flask 250 mg of ilomastat is added to 11 mL of ethanol and mixed until dissolved.

A clean, dried stent is then sprayed with PVP until a smooth confluent polymer layer was achieved. The stent was then dried in a vacuum oven at 50° C. for 30 minutes.

Next, successive layers of ilomastat are applied to the polymer-coated stent. The stent is allowed to dry between each of the successive ilomastat coats. After the final ilomostat coating has dried, three successive coats of PVP are applied to the stent followed by drying the coated stent in a vacuum oven at 50° C. over night. The dried, coated stent is weighed and its weight recorded.

The concentration of drug in the drug/polymer solution and the final amount of drug loaded onto the stent determine the final coating weight. Final coating weight is calculated by subtracting the stent's pre-coating weight from the weight of the dried, coated stent.

### Example 4

### Coating a Cleans Dried Stent with Pure Drug

1.00 g of ilomastat is carefully weighed and added to a small neck glass bottle containing 12 ml of ethanol. The ilomastat-ethanol suspension is then heated at 50° C. for 15 minutes and then mixed until the ilomastat is completely dissolved.

Next a clean, dried stent is mounted over the balloon portion of angioplasty balloon catheter assembly. The stent is then sprayed with, or in an alternative embodiment, dipped into, the ilomastat-ethanol solution. The coated stent is dried in a vacuum oven at 50° C. over night. The dried, coated stent was weighed and its weight recorded.

The concentration of drug loaded onto (into) the stents is determined based on the final coating weight. Final coating weight is calculated by subtracting the stent's pre-coating weight from the weight of the dried, coated stent.

### Example 5

### Abdominal Aneurysm

In one embodiment, a stent loaded with at least one of ilomastat or PG-530742 can be used to deliver the MMP inhibitor locally to the abdominal aorta for treatment/stabilization of an abdominal aneurysm.

### Example 6

### Local Delivery to Coronary Artery

In one embodiment, a stent loaded with at least one of ilomastat or PG-530742 can be used to deliver the MMP inhibitor locally to the coronary artery for the combined treatment of restenosis and atherosclerotic plaque stabilization.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A medical device for treating a vascular condition comprising:
a stent;
at least one polymer; and
a therapeutically effective amount of at least one matrix metalloproteinase inhibitor;
wherein said stent is adapted to deliver said matrix metalloproteinase inhibitor to a tissue within a mammal suffering from a vascular condition.

2. The medical device according to claim 1, wherein said matrix metalloproteinase inhibitor comprises 3-(N-hydroxycarbamoyl)-2(R)-isobutylpropionyl-L-tryptophan methylamide (ilomostat).

3. The medical device according to claim 2, wherein said 3-(N-hydroxycarbamoyl)-2(R)-isobutylpropionyl-L-tryptophan methylamide is present in an amount of from about 1 to about 1000 μg.

4. The medical device according to claim 1, wherein said matrix metalloproteinase inhibitor comprises 2-[4-(4-methoxybenzamido)phenylsulfonamido]-6-(4-morpholinyl)-4-hexynoic acid (PG-530742).

5. The medical device according to claim 4, wherein said 2-[4-(4-methoxybenzamido)phenylsulfonamido]-6-(4-morpholinyl)-4-hexynoic acid is present in an amount of from about 1 to about 1000 μg.

6. The medical device according to claim 1, wherein said vascular condition is selected from the group consisting of plaque rupture, aneurysm, stenosis, restenosis, atherosclerosis, and combinations thereof.

7. The medical device according to claim 1, wherein said polymer is selected from the group consisting of polyurethanes, silicones, polyolefins, polyisobutylene, ethylene-alphaolefin copolymers, acrylic polymers and copolymers, ethylene-co-vinylacetate, polybutylmethacrylate, vinyl halide polymers and copolymers, polyvinyl chloride; polyvinyl ethers, polyvinyl methyl ether, polyvinylidene halides, polyvinylidene fluoride, polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate; cellophane, cellulose nitrate, cellulose propionate, cellulose ethers, carboxymethyl cellulose, poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes, biomolecules such as fibrin, fibrinogen, cellulose, starch, collagen and hyaluronic acid, and combinations thereof.

8. The medical device according to claim 1, wherein said stent comprises a ratio of matrix metalloproteinase inhibitor to polymer.

9. The medical device according to claim 8, wherein said ratio is between about 1:1 and about 1:20.

10. A vascular stent comprising a polymeric coating having a therapeutically effective amount of at least one matrix metalloproteinase inhibitor.

11. The vascular stent of claim 10, further comprising a primer coat.

12. The vascular stent of claim 10, wherein said matrix metalloproteinase inhibitor comprises 3-(N-hydroxycarbamoyl)-2(R)-isobutylpropionyl-L-tryptophan methylamide (ilomastat).

13. The vascular stent of claim 10, wherein said matrix metalloproteinase inhibitor comprises 2-[4-(4-methoxybenzamido)phenylsulfonamido]-6-(4-morpholinyl)-4-hexynoic acid (PG-530742).

14. The vascular stent of claim 10, wherein said polymeric coating comprises at least one polymer selected from the group consisting of polyurethanes, silicones, polyolefins, polyisobutylene, ethylene-alphaolefin copolymers, acrylic polymers and copolymers, ethylene-co-vinylacetate, polybutylmethacrylate, vinyl halide polymers and copolymers, polyvinyl chloride; polyvinyl ethers, polyvinyl methyl ether, polyvinylidene halides, polyvinylidene fluoride, polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate; cellophane, cellulose nitrate, cellulose propionate, cellulose ethers, carboxymethyl cellulose, poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes, biomolecules such as fibrin, fibrinogen, cellulose, starch, collagen and hyaluronic acid, and combinations thereof.

15. The vascular stent of claim 10, wherein said stent comprises a ratio of matrix metalloproteinase inhibitor to polymer.

16. The vascular stent of claim 15, wherein said ratio is between about 1:1 and about 1:20.

17. A method of treating a vascular condition in a mammal comprising local delivery of at least one matrix metalloproteinase inhibitor to a mammal suffering from a vascular condition selected from the group consisting of plaque rupture, aneurysm, stenosis, restenosis, atherosclerosis, and combinations thereof.

18. The method according to claim 17, wherein said matrix metalloproteinase inhibitor is delivered using a vascular stent.

19. The method according to claim 17, wherein said matrix metalloproteinase inhibitor comprises 3-(N-hydroxycarbamoyl)-2(R)-isobutylpropionyl-L-tryptophan methylamide (ilomastat)

20. The method according to claim 17, wherein said matrix metalloproteinase inhibitor comprises 2-[4-(4-methoxybenzamido)phenylsulfonamido]-6-(4-morpholinyl)-4-hexynoic acid (PG-530742).

21. A method for inhibiting restenosis comprising providing a vascular stent having a coating comprising an therapeutically effective amount of a bioactive agent selected from the group consisting of 3-(N-hydroxycarbamoyl)-2(R)-isobutylpropionyl-L-tryptophan methylamide, 2-[4-(4-methoxybenzamido)phenylsulfonamido]-6-(4-morpholinyl)-4-hexynoic acid, and combinations thereof.
